(19) United States
(12) Patent Application Publication
(10) Pub. No.: US 2015/0328149 A1
(43) Pub. Date: Nov. 19, 2015

(54) Method for Administering an Antibiotic Dosage

(71) Applicant: Dianne Emler, Magnolia, TX (US)

(72) Inventor: Dianne Emler, Magnolia, TX (US)

(21) Appl. No.: 14/280,493

(22) Filed: May 16, 2014

(51) Int. Cl. A61K 9/00; A61K 31/5578; A61K 45/06

(52) U.S. Cl. CPC A61K 9/0053; A61K 45/06; A61K 31/5578

(57) ABSTRACT

A method of administering an antibiotic dosage by supplementing the antibiotic with anti-viral, mucosin, bacterial retardants, and other compounds, and delivering the compounds via direct methods to infection sites to allow lower dosage in localized areas with less effect on the rest of the body.

### BACKGROUND OF THE INVENTION

1. Field of the Invention

This disclosure relates to the field of drug delivery, particularly to the delivery of antibiotics in a direct route to infection sites.

2. BACKGROUND OF THE INVENTION

Antibiotics are typically delivered orally or via injection into the bloodstream. Orally delivered antibiotics enter the bloodstream via the digestive system. The antibiotic, or its metabolites, are dispersed throughout the patient's body. In some cases this may be desirable, but in many cases an infection is highly localized. In such cases, antibiotic delivery to the locations of the infection may be more effective at lower doses. The antibiotic may also have a much quicker effect and improved efficiency. In some cases, antibiotics may be adversely affected by the conditions of the digestive track or may be tempered by other bodily systems in route to an infection site.

### BRIEF DESCRIPTION OF THE DRAWINGS

FIG. 1 illustrates a multilayer tablet containing three compounds in accordance with an exemplary embodiment of the invention.

FIG. 2 illustrates a multi-component capsule containing three compounds in accordance with an exemplary embodiment of the invention.

FIG. 3 shows a foley catheter with antibiotic coating in accordance with an exemplary embodiment of the invention.

FIG. 3A shows the foley catheter of FIG. 3 with an inflated balloon tip in accordance with an exemplary embodiment of the invention.

FIG. 4 shows the use of a foley on a male patient.

FIG. 4A shows a foley catheter properly positioned in the bladder of a patient in accordance with an exemplary embodiment of the invention.

FIG. 5 shows a patient administering an inhalation treatment in accordance with an exemplary embodiment of the invention.

### DETAILED DESCRIPTION OF THE PREFERRED EMBODIMENTS

Desired is delivery of an antibiotic compound which results in more antibiotic reaching the infection site, and less throughout the rest of the body during initial dosing. As infections are sites of other disease and foreign bodily invasion due to localized body resistance, the antibiotics can be supplemented with bacterial retardants and antivirals. A more direct administration of antibiotics and other compounds to an infection site would allow faster response times to the medications, lower effective dosages with less impact on the rest of the body.

Common illnesses often cause mucus glands to increase production as part of a defense mechanism. This thicker mucus can trap drugs from reaching the site of desired impact. This mucus is meant to protect the body by trapping bacteria or viruses. However, it also traps antivirals and antibiotics as well, preventing them from reaching the mucosal glands where infection may reside. Therefore, it is desirable to include mucosin compounds with the antibiotic and antivirals to help them reach the infection through the body's defenses.

In another embodiment, an antibiotic is combined with other medicinal compounds. In one embodiment, the antibiotic is combined with a bacterial retardant. Many bacterial retardants are commonly known, such as 2-isopropyl-5-methylphenol (commonly known as Thymol) and Carvacrol or Cymophenol, which may assist the antibiotic by weakening bacterial strains. The bacterial retardant may further prevent bacterial re-infection. In one embodiment, antibiotic and bacterial retardant may work to remove the bacterial infection from a wound site while other compounds may prevent re-infection or otherwise promote healing properties and conditions.

An exemplary embodiment relates to an antibiotic delivery device which involves the enrobing of one compound with another in a layered configuration to aid the compounds in bypassing bodily defenses. For instance, an antibiotic may be utilized as a core which is surrounded by an anti-viral or bacterial retardant. In addition, the anti-viral or bacterial retardant may be further surrounded by a third layer consisting of a mucosin. This muli-part compound may be a single dosage tablet, or may be further contained in capsules. The components may be layered such that the first bodily defenses are encountered by the outer compounds, and later defenses are encountered by more internal compounds. In another embodiment, the layers may be alternated such that fresh compounds are exposed at different release points during the dosage process. One skilled in the arts would appreciate that the materials in each layer and the number of layers can be configured for specific applications depending on the resistance expected.

Another exemplary embodiment relates to an antibiotic delivery device that provides the antibiotic in a form adapted for inhalation into the patient's lungs. In this embodiment, a solid form may be rendered into nanoparticles which are inhaled into the respiratory track. In another embodiment, the antibiotics can be dissolved into solution then atomized and inhaled into the respiratory track. Either method results in the antibiotic compound being delivered directly to the respiratory track where it can immediately fight infections of the respiratory system. This direct delivery method results in faster delivery and more concentration at the site of infection, allowing for lower overall dosage. One skilled in the art would appreciate that the antibiotic delivery to the patient's respiratory system may be via inhaling of an aerosol, gas, mist, or vapor and can be done with an aerosol inhaler or a nebulizer.

In the preceding examples, the liquid medication used in an inhaler or nebulizer may be the drug that is itself a liquid, or it may be a solution, suspension, or emulsion that contains the medicates of interest. In the preferred embodiment, the liquid medicament is a combination of the antibiotic which is mixed along with one or more of the described enhancers into a solution, a suspension, or an emulsion.

FIG. 1 illustrates a multilayer tablet containing three compounds in accordance with an exemplary embodiment of the invention. The tablet (100) comprises a plurality of layers (150, 160, and 170) each of which is a different medicate compound. The actual arrangement of the layers are determined by the type of bodily defenses expected for a specific medical ailment to be encountered by the medicine. By layering the medicines, their release can be controlled to maximize the effectiveness of each by timing their release to different timing in the body. Figure one shows a first medicinal compound (150) enrobed within a second medicinal compound (160) and further enrobed within a third medicinal compound (170). This is illustrated within a single tablet. One skilled in the art would appreciate that different compounds could be layered in different levels and that the enrobing may comprise other compounds which may hasten or slow the absorption process. Further, a tablet may contain several individual portions which may have different layered components or the same layered components in different orders, thicknesses, and/or compositions.

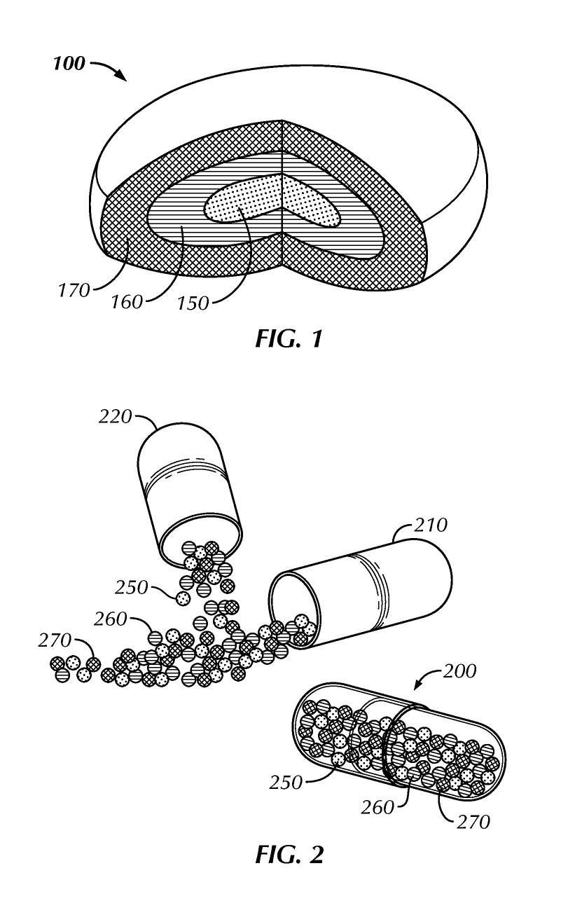

FIG. 2 illustrates a multi-component capsule containing three compounds in accordance with an exemplary embodiment of the invention. In the preferred embodiment, a capsule is assembled with multiple medicinal compounds contained within. The typical two-piece gel capsule has a telescoping shell comprising a sleeve (210) and a cap (220). Within the capsule (200) dosages of drugs may be contained in powdered or crystalline form. In the preferred embodiment, the capsule (200) contains an antibiotic, anti-viral, and a mucosin. The compounds may be in the crystalline form and formed into three distinct compounds (250, 260, 270). Each compound may contain a single compound, or may be a layer or mix of more than one compound. One skilled in the arts would appreciate that binders, disintegrates, or other agents may be used to keep the medicine in crystallized form and to control dosaging and release times while still being within the spirit of the teaching.

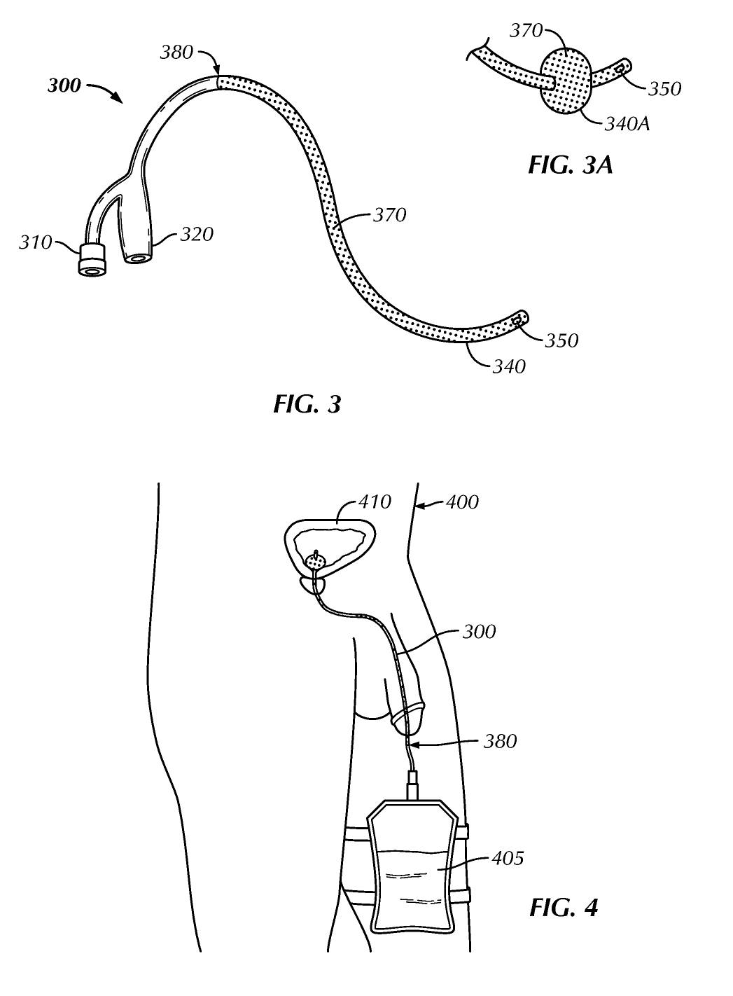

FIG. 3 shows a foley catheter with antibiotic coating in accordance with an exemplary embodiment of the invention. Use of a foley catheter necessarily involves inserting a foreign object into the patient. This introduction of a foreign object is a likely source for contamination and infection. Therefore, it is desireable to supplement the catheter with an antibiotic coating which can prevent bacterial infection. By coating the catheter with antibacterial at least to the point of insertion, the patient receives a dosage of medication which can fight infection, and reduce the initial chance of infestation of the site. In the preferred embodiment, the medicinal coating comprises at least an antibiotic, an antiviral, a mucosin, and/or a bacterial retardant, or a combination of one or more of the compounds. A Foley catheter (300) is a dual lumen catheter having at an external end, a catheter balloon port (310), and a catheter urine drainage port (320) which joins to a bladder. The distal end of the catheter (300) comprises a balloon (340) and a bladder opening (350). In the preferred embodiment, a coating of medicinal compounds is layered (370) on the outer surface of the catheter (300) at least from the bladder opening (350) back toward the ports (310, 320) to the point at which the catheter would exit the body (380). In some embodiments, the medicinal compounds may be infused with the catheter's body structure or may be impregnated into the material comprising the catheter body.

FIG. 3A shows the foley catheter of FIG. 3 with an inflated balloon tip in accordance with an exemplary embodiment of the invention. Once the foley catheter (300) is inserted into the urethra opening, the balloon (340A) is inflated to prevent the catheter from being removed accidentally. The act of inflating the balloon disturbs the medicinal coating on the catheter and begins the dissolution process of the medicinal compounds in the bodily fluids.

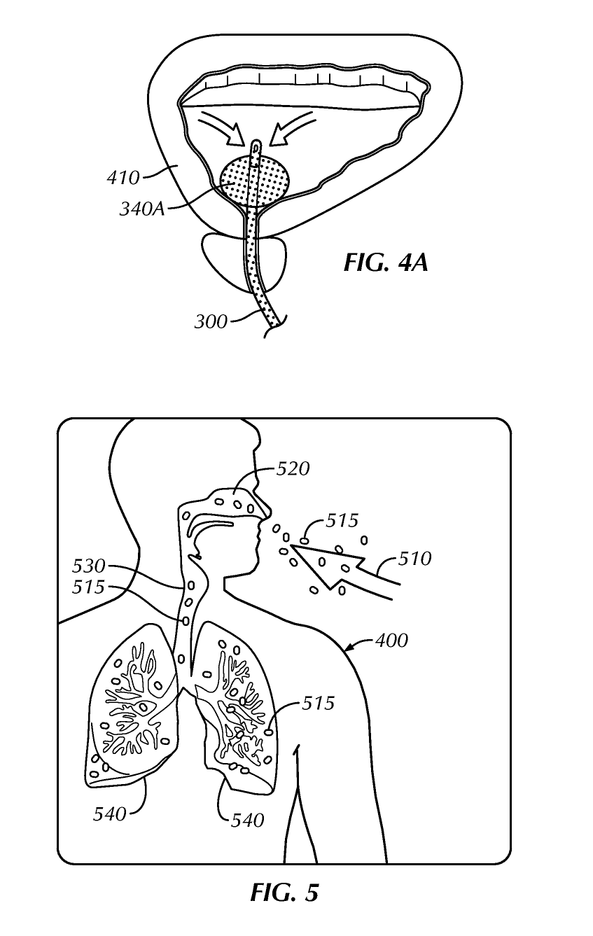

FIG. 4 shows the use of a foley on a male patient. The catheter (300) is inserted into the urethra opening of the patient (400) to the point of medicinal coating (380) so the bladder opening of the catheter (300) reaches the bladder (410). The port end of the catheter is then connected to a bladder (405) to catch the bodily fluids. FIG. 4A shows a foley catheter properly positioned in the bladder of a patient in accordance with an exemplary embodiment of the invention. The catheter (300) is inserted into the bladder (410) and the balloon (340A) is inflated to secure it.

FIG. 5 shows a patient administering an inhalation treatment in accordance with an exemplary embodiment of the invention. The Patient (400) inhales (510) particles (515) of medicinal compounds into the nasal passages (520) to pass through the trachea (530) and into the lungs (540). In the lungs (540), the compounds (515) are deposited into the air sacs where infections are often located.

The diagrams in accordance with exemplary embodiments of the present invention are provided as examples and should not be construed to limit other embodiments within the scope of the invention. Drawings may not be to scale and should not be construed to limit the invention to the particular proportions illustrated. Additionally, some elements illustrated in the singularity may actually be implemented in a plurality. Further, some element illustrated in the plurality could actually vary in count. Further, some elements illustrated in one form could actually vary in detail. Further yet, specific numerical data values (such as specific quantities, numbers, categories, etc.) or other specific information should be interpreted as illustrative for discussing exemplary embodiments. Such specific information is not provided to limit the invention.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

What is claimed is:

1. A method of antibiotic delivery comprising;
delivering an antibiotic compound to an infection site which is not externally presented.

2. A method of antibiotic delivery, as described in claim 1, wherein the delivery is by non-blood borne transmission directly to an infection site which is not externally presented.

3. A method of antibiotic delivery, as described in claim 1, wherein the antibiotic compound further comprises a bacterial retardant.

4. A method of antibiotic delivery, as described in claim 1, wherein the antibiotic compound further comprises an antiviral.

5. A method of antibiotic delivery, as described in claim 1, wherein the antibiotic compound further comprises a mucosin.

6. A method of antibiotic delivery, as described in claim 1, wherein the antibiotic is in a liquid form.

7. A method of antibiotic delivery, as described in claim 6, wherein the antibiotic, in liquid form, is inserted into an infected bladder via a catheter.

8. A method of antibiotic delivery, as described in claim 6, wherein the antibiotic, in liquid form is administered by bolus injection to the infected area.

9. A method of antibiotic delivery, as described in claim 6, wherein the antibiotic is atomized into an aerosol and inhaled into the respiratory passages.

10. A method of antibiotic delivery, as described in claim 1, wherein the antibiotic is in a granular solid form.

11. A method of antibiotic delivery, as described in claim 10, wherein the antibiotic granules are enclosed in one or more layers of an bacterial retardant, an antiviral, and/or a mucosin.

12. A method of antibiotic delivery, as described in claim 1, wherein the antibiotic compound further comprises a bacterial retardant, an antiviral, and/or a mucosin.

13. A method of manufacturing an antibiotic compound comprising:
taking antibiotic in solid granular form;
enrobing an antiviral; and
enrobing bacteria retardant.

14. A method of manufacturing an antibiotic compound, as described in claim 15 wherein the granules are further enrobed in mucosin.

15. A method of manufacturing an antibiotic compound, as described in claim 15 wherein the antibiotic, antiviral, and bacteria retardant, are each in granular form and are compressed into a pill or capsule.

16. A method of antibiotic delivery comprising;
delivering an antibiotic compound to an infection site wherein the infection site is externally present.

17. A method of antibiotic delivery, as described in claim 16, wherein the antibiotic is in a powdered form.

18. A method of antibiotic delivery, as described in claim 17, wherein the antibiotic further comprises a bacterial retardant and/or an antiviral.

19. A method of antibiotic delivery, as described in claim 16, wherein the antibiotic is in a liquid form.

20. A method of antibiotic deliver, as describe in claim 19 wherein the antibiotic further comprises a bacterial retardant and/or an antiviral.
